(19) United States
(12) Patent Application Publication
(10) Pub. No.: US 2022/0175291 A1
(43) Pub. Date: Jun. 9, 2022

(54) SMART CLOTHING FOR SENSING HEART PHYSIOLOGICAL ACTIVITIES AND LUNG RESPIRATORY CONDITIONS

(71) Applicant: National Cheng Kung University, Tainan City (TW)

(72) Inventors: SHUENN-YUH LEE, Tainan City (TW); Yi-Wen Hung, Kaohsiung City (TW); PO-HAN SU, Chiayi County (TW)

(21) Appl. No.: 17/129,194

(22) Filed: Dec. 21, 2020

(30) Foreign Application Priority Data
Dec. 4, 2020 (TW) 109142725

(51) Int. Cl. A61B 5/256; A61B 5/273; A61B 5/0205; A61B 5/277; A61B 5/332; A61B 5/00; A41D 1/00

(52) U.S. Cl. CPC A61B 5/256; A61B 5/273; A61B 5/0205; A61B 5/02438; A61B 5/332; A61B 5/6804; A41D 1/002; A61B 5/277

(57) ABSTRACT

A smart clothes for sensing heart physiological activities and lung respiratory conditions is provided, the smart clothes utilizes conductive connecting elements for being externally connected to a control module, such that the control module can be expanded or upgraded according to functional requirements. Further in the smart clothes, sensing elements and signal transmission wires are made of conductive fabric. As the conductive fabric sensing elements and signal transmission wires are well attached to a clothing body of the smart clothing, the sensing elements can be better adhered to human skin, and thereby sensing accuracy is improved.

### CROSS-REFERENCE TO RELATED APPLICATIONS

This application claims the priority of Republic of China Patent Application No.109142725 filed on Dec. 4, 2020, in the State Intellectual Property Office of the R.O.C., the disclosure of which is incorporated herein by reference.

### BACKGROUND OF THE INVENTION

### Field of the Invention

The present invention relates to smart clothing, and more particularly, to smart clothing for sensing heart physiological activities and lung respiratory conditions, which allows a user to have overall health monitoring in daily life or during exercise.

### Descriptions of the Related Art

Innovations of medical technology introduce various sensing devices for sensing physiological signals of users nowadays, especially smart wearable devices that are getting popular and widely applied in various areas, for example, smart sport watch or wristband. Moreover, due to maturation of application technology such as Internet of Things (IoT), cloud with big data and Artificial Intelligence (AI), sport smart clothing becomes available and can be used to collect exercise data such as heart rate data, moving distance, respiration rate, and so on.

Conventional sport smart clothing however easily makes a user uncomfortable when wearing it because of its hardware structural limitation, which adversely affects accuracy of physiological signals being collected. For example, electrocardiogram (ECG) data are commonly used in cardiovascular disease analysis and diagnosis. Electric potential change of a heart itself reflects on human skin through conductive tissue around the heart and body fluids, and microelectrode technology is utilized to record potential difference between inside and outside of myocardial cells induced by tiny electric impulse change of the heart, that is to record electric potential change during a heart rhythm cycle, so as to collect results of multiple ECG leads. Generally, a plurality of electrodes and wires connected thereto are placed on body skin of the user, and all the wires are connected to a recorder to continuously monitor ECG signals of the multiple leads. However, the wires and recorder carried by the user usually bother the user's daily life, and noise is easily generated by any movement of the wires and recorder, making it difficult to analyze and read the signals. Further, it is not handy for the user to wear and remove the plurality of electrodes, wires or recorder, not to mention a trouble of cleaning them after use, such that the conventional smart clothing is not welcome.

Therefore, how to solve the above issues of the conventional smart clothing, such as wearing inconvenience, difficulty in cleaning and poor sensing accuracy when collecting physiological signals, is an important task to work on in the art.

### SUMMARY OF THE INVENTION

In view of the above drawbacks in the prior art, a primary object of the present invention is to provide smart clothing for sensing heart physiological activities and lung respiratory conditions, which allows a user to easily wear it like normal clothes, and allows physiological signals to be sensed regardless of the user's movement, so as to achieve effects of desirable adhesion to human skin and good sensing accuracy.

Another object of the present invention is to provide smart clothing for sensing heart physiological activities and lung respiratory conditions, wherein its sensing elements are all made of soft fabric to give a user great comfort and are washable together with the clothing itself, such that the smart clothing is easy to clean and durable.

According to the above purpose and another purpose of the present invention, the present invention is to provide a smart clothing for sensing heart physiological activities, the smart clothing for sensing heart physiological activities including: a clothing body having a contact surface and an outer surface opposite to the contact surface, wherein the contact surface is for contacting human skin; an electrocardiogram lead collector including at least one lead collecting structure mounted on the contact surface of the clothing body, wherein the lead collecting structure includes a pair of electrodes, and lead wires respectively connected to the electrodes, with the electrodes and lead wires being made of conductive fabric, wherein each of the lead wires is formed with a first conductive connecting element at an end thereof extended in a predetermined direction, and the first conductive connecting element has a connecting end penetrating the end of each of the lead wires and the clothing body to be placed on the outer surface of the clothing body; and a control module including a substrate formed with a plurality of functional units and mounted on the outer surface of the clothing body, wherein the substrate is formed with a second conductive connecting element on a side thereof for being connected to the connecting end of the first conductive connecting element, so as to allow the plurality of functional units of the substrate to process heart physiological activity signals sensed by the electrocardiogram lead collector.

Preferably, in the smart clothing for sensing heart physiological activities said above, wherein the substrate is further formed with a pair of third conductive connecting elements on the side thereof, and the smart clothing further includes: a respiration converter formed of a multi-layer structure, for outputting capacitance change signals corresponding to human body cavity undulation during breathing, wherein the multi-layer structure includes: an upper conductive fabric layer, a lower conductive fabric layer, an insulation film provided between the upper conductive fabric layer and the lower conductive fabric layer, at least one upper wire connected to the upper conductive fabric layer, and at least one lower wire connected to the lower conductive fabric layer, and the multi-layer structure is mounted on the contact surface or the outer surface of the clothing body, wherein the upper wire and the lower wire are made of conductive fabric, and each of the upper and lower wires is formed with a fourth conductive connecting element at an end thereof extended in the predetermined direction, and the fourth conductive connecting element is connected to the pair of third conductive connecting elements, so as to allow the control module to process the capacitance change signals outputted by the respiration converter and sense human body respiratory conditions.

Preferably, in the smart clothing for sensing heart physiological activities said above, further including: a container bag attached to the outer surface of the clothing body, for accommodating the control module ; and the smart clothing further including: a first coupling element formed on the outer surface of the clothing body, wherein the container bag has a movable side formed with a second coupling element for being coupled to the first coupling element, and coupling between the first coupling element and the second coupling element allows the movable side to be lifted or closed in order to detach or mount the control module.

Moreover, the present invention further provides a smart clothing for sensing lung respiratory conditions, including: a clothing body having a contact surface and an outer surface opposite to the contact surface, wherein the contact surface is for contacting human skin; a respiration converter formed of a multi-layer structure, for outputting capacitance change signals corresponding to human body cavity undulation during breathing, wherein the multi-layer structure includes: an upper conductive fabric layer, a lower conductive fabric layer, an insulation film provided between the upper conductive fabric layer and the lower conductive fabric layer, at least one upper wire connected to the upper conductive fabric layer, and at least one lower wire connected to the lower conductive fabric layer, and the multi-layer structure is mounted on the contact surface or the outer surface of the clothing body, wherein the upper wire and the lower wire are made of conductive fabric, and each of the upper and lower wires is formed with a first conductive connecting element at an end thereof extended in the predetermined direction; and a control module including a substrate formed with a plurality of functional units, wherein the substrate is formed with a second conductive connecting element on a side thereof for being connected to the connecting end of the first conductive connecting element, and the substrate is mounted on the contact surface or the outer surface of the clothing body, so as to allow the plurality of functional units of the substrate to process the capacitance change signals outputted by the respiration converter and sense human body respiratory conditions.

Preferably, in the smart clothing for sensing lung respiratory conditions said above, further including: a container bag attached to the outer surface of the clothing body, for accommodating the control module; and the smart clothing for sensing lung respiratory conditions further including: a first coupling element formed on the outer surface of the clothing body, wherein the container bag has a movable side formed with a second coupling element for being coupled to the first coupling element, and coupling between the first coupling element and the second coupling element allows the movable side to be lifted or closed in order to detach or mount the control module.

Preferably, in the smart clothing for sensing lung respiratory conditions said above, wherein the substrate is further formed with a pair of third conductive connecting elements on the side thereof, and the smart clothing further includes an electrocardiogram lead collector, the electrocardiogram lead collector including: at least one lead collecting structure mounted on the contact surface of the clothing body, wherein the lead collecting structure includes a pair of electrodes, and lead wires respectively connected to the electrodes, with the electrodes and lead wires being made of conductive fabric, wherein each of the lead wires is formed with a fourth conductive connecting element at an end thereof extended in a predetermined direction, and the fourth conductive connecting element has a connecting end penetrating the end of each of the lead wires and the clothing body to be placed on the outer surface of the clothing body.

Advantageously the present invention utilizes conductive connecting elements such as buttons for being externally connected to a control module, such that the control module can be expanded or upgraded according to functional requirements. Further in the present invention, sensing elements and signal transmission wires are made of conductive fabric. This solves the problems of wearing inconvenience and difficulty in cleaning caused by installing transmission functional modules in the conventional smart clothing. As the conductive fabric sensing elements and signal transmission wires are well attached to a clothing body of the smart clothing, the sensing elements can be better adhered to human skin, and thereby sensing accuracy is improved.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a schematic diagram showing appearance of smart clothing for sensing heart physiological activities according to a preferred embodiment of the present invention.

FIG. 2 is a side exploded view showing lead wires and a control module of the smart clothing according to the present invention.

FIG. 3 is a basic architectural diagram showing a plurality of functional units of the control module of the smart clothing for sensing heart physiological activities according to the present invention.

FIG. 4 is a schematic diagram showing appearance of a contact surface of a clothing body of the smart clothing for sensing heart physiological activities according to another preferred embodiment of the present invention.

FIG. 5A and FIG. 5B are schematic diagrams showing a clothing body of the smart clothing for sensing heart physiological activities according to another preferred embodiment of the present invention.

FIG. 6 is a schematic diagram showing a clothing body of the smart clothing for sensing heart physiological activities according to another preferred embodiment of the present invention.

FIG. 7 is a schematic diagram showing appearance of smart clothing applied to sensing lung respiratory conditions in the present invention.

FIG. 8 is a structural cross-sectional diagram of a respiration converter of the smart clothing for sensing lung respiratory conditions according to the present invention.

FIG. 9A and FIG. 9B are basic architectural diagrams showing a plurality of functional units of a control module of the smart clothing for sensing lung respiratory conditions according to the present invention.

FIG. 10 is a schematic diagram showing appearance of smart clothing for sensing heart physiological activities and lung respiratory conditions according to the present invention.

### DETAILED DESCRIPTION OF THE PREFERRED EMBODIMENT

Embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In the drawings, the shapes and dimensions of elements may be exaggerated for clarity, and the same reference numerals will be used throughout to designate the same or like components.

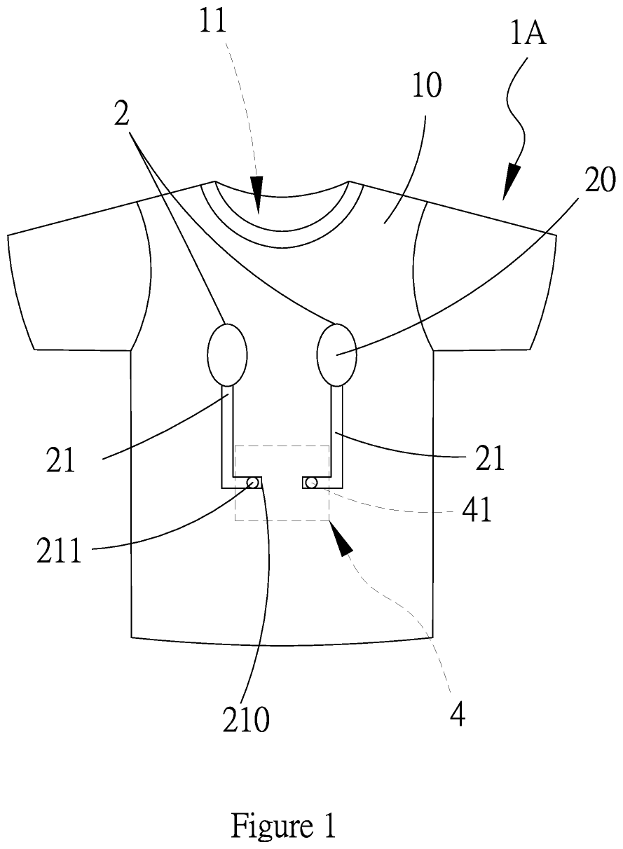

The present invention provides smart clothing for sensing heart physiological activities and lung respiratory conditions, which is easy to wear, comfortable and durable. Firstly, as shown in FIG. 1, which is a schematic diagram showing appearance of smart clothing for sensing heart physiological activities according to a preferred embodiment of the present invention, the smart clothing for sensing heart physiological activities in this embodiment includes: a clothing body 1A, an electrocardiogram (ECG) lead collector 2 and a control module 4. The clothing body 1A has a contact surface 10 and an outer surface 11 opposite to the contact surface 10, wherein the contact surface 10 is used for touching human skin. For easy understanding, the clothing body 1A is inside out in FIG. 1 with the contact surface 10 shown outside. The ECG lead collector 2 includes at least one lead collecting structure mounted on the contact surface 10 of the clothing body 1A, wherein the at least one lead collecting structure includes a pair of electrodes 20, and lead wires 21 respectively connected to the electrodes 20. It should be noted that, the smart clothing for sensing heart physiological activities in FIG. 1 shows single lead ECG sensing in this embodiment, which is merely to simplify drawings and description, and does not limit implementations of the present invention. It is flexible to mount a plurality of pairs of electrodes 20 on the contact surface 10 of the clothing body 1A to provide multiple (three, six or twelve) leads ECG sensing according to different implementations.

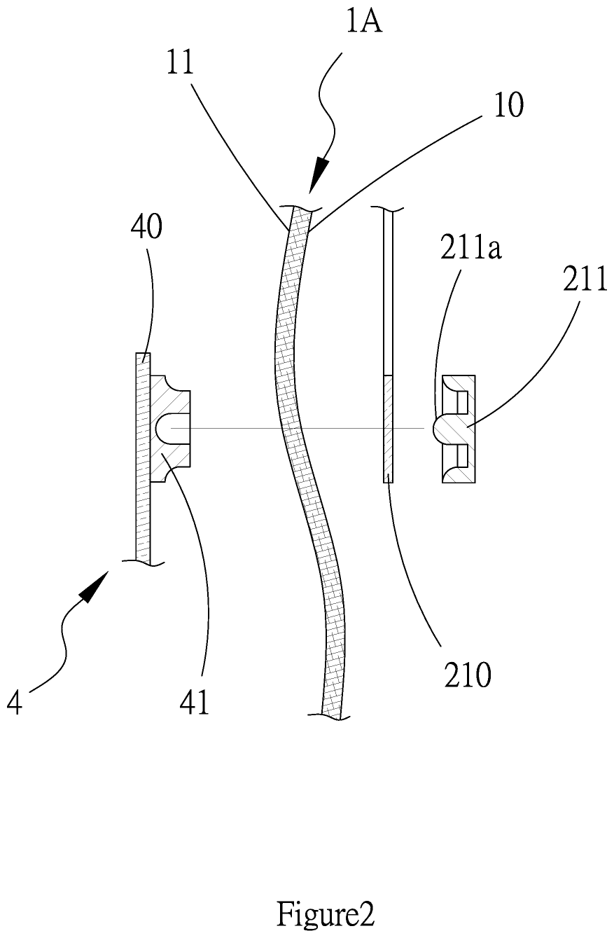

The electrodes 20 and lead wires 21 are made of conductive fabric. Each of the lead wires 21 has an end 210 extended in a predetermined direction and formed with a conductive connecting element 211, wherein the predetermined direction is a direction toward the control module 4. Electric signals sensed by the electrodes 20 are sent to the control module 4 by the lead wires 21, wherein a connecting end 211a of the conductive connecting element 211 penetrates the end 210 of each of the lead wires 21 and the clothing body 1A to be mounted on the outer surface 11 of the clothing body 1A, and the connecting end 211a of the conductive connecting element 211 is connected to the control module 4. The control module 4 is a substrate 40 formed with a plurality of functional units. The substrate 40 is placed on the outer surface 11 of the clothing body 1A, and a conductive connecting element 41 is provided on a side of the substrate 40 and used to be connected to the conductive connecting element 211 of each of the lead wires 21, as shown in FIG. 2, so as to allow the plurality of functional units of the substrate 40 to process heart physiological activity signals sensed by the ECG lead collector 2. It should be noted that, the conductive connecting element 41 on the substrate 40 and the conductive connecting element 211 penetrating the end 210 of each of the lead wires 21 and the clothing body 1A, can be made in the form of buttons such as male buttons or female buttons. FIG. 2 shows that the conductive connecting element 211 is a male button and the conductive connecting element 41 is a female button. The present invention is not limited to this configuration. Alternatively, the conductive connecting element 211 can be a female button and the conductive connecting element 41 can be a male button. Further, the conductive connecting elements are not limited to buttons but can be implemented in other metal or medical connectors.

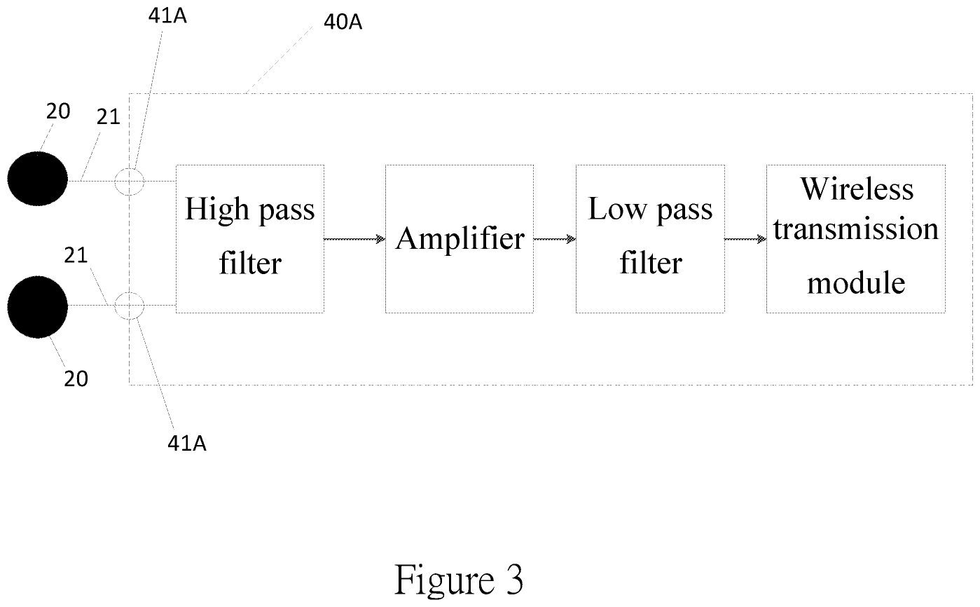

Referring to FIG. 3, which is a basic architectural diagram showing a plurality of functional units of the control module of the smart clothing for sensing heart physiological activities according to the present invention, this embodiment is implemented with a control module 40A for providing ECG sensing processing. The control module 40A at least includes: a high pass filter, an amplifier, a low pass filter and a wireless transmission module. Electric signals generated by heart activities are transferred to human skin through body tissue. The electrodes 20, lead wires 21 and conductive connecting elements 41A allow the electric signals to be further transmitted to the control module 40A. The high pass and low pass filters remove unwanted signals outside ECG signal frequency bands, and the amplifier and low pass filter with gain are used to amplify ECG signals, for allowing the wireless transmission module to take a sample of the signals and transmit it to a monitoring device where the sampled signals are processed by such as analysis, computation and so on. The monitoring device is for example, personal digital assistant, mobile phone, computer or network server, etc. and is allowed to analyze heart diseases or other health-related information through algorithms such as artificial intelligence.

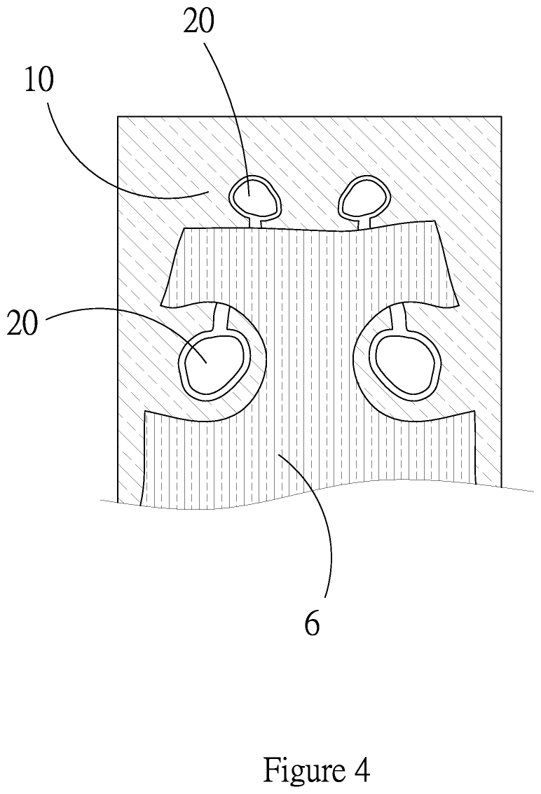

FIG. 4 shows another embodiment of the smart clothing for sensing heart physiological activities. In this embodiment, the smart clothing further includes a spacer 6 provided on part of the contact surface 10 of the clothing body, for covering the lead wires and isolating the lead wires from human skin, with the electrodes 20 being exposed from the spacer 6.

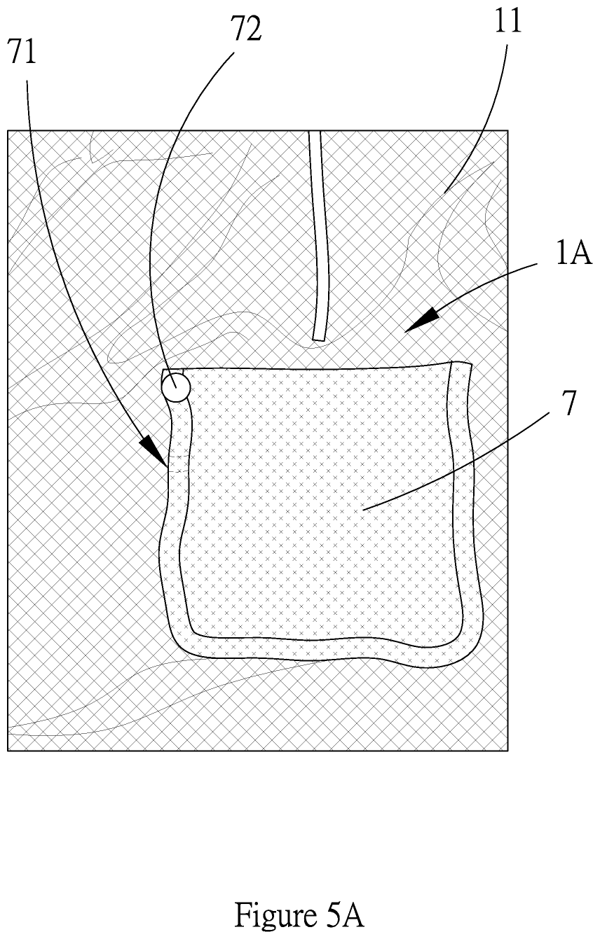

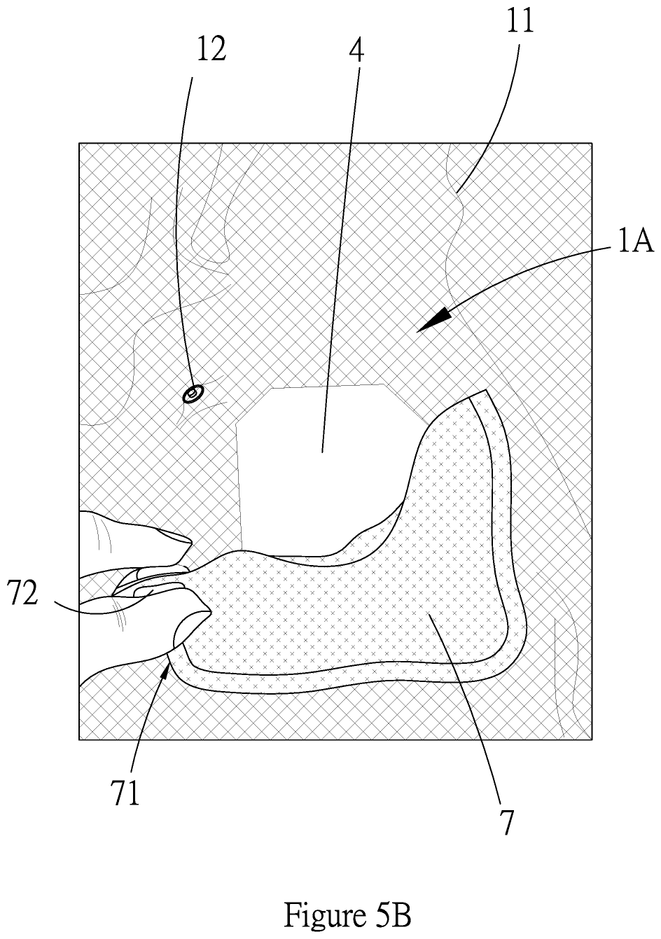

FIG. 5A and FIG. 5B show another embodiment of the smart clothing for sensing heart physiological activities. In this embodiment, the smart clothing further includes a container bag 7 attached to the outer surface 11 of the clothing body 1A, for accommodating the control module 4. Further on the outer surface 11 of the clothing body 1A there is formed a first coupling element 12, and the container bag 7 has a movable side 71 formed with a second coupling element 72 for being coupled to the first coupling element 12. Coupling between the first coupling element 12 and the second coupling element 72 allows the movable side 71 to be lifted or closed in order to detach or mount the control module 4.

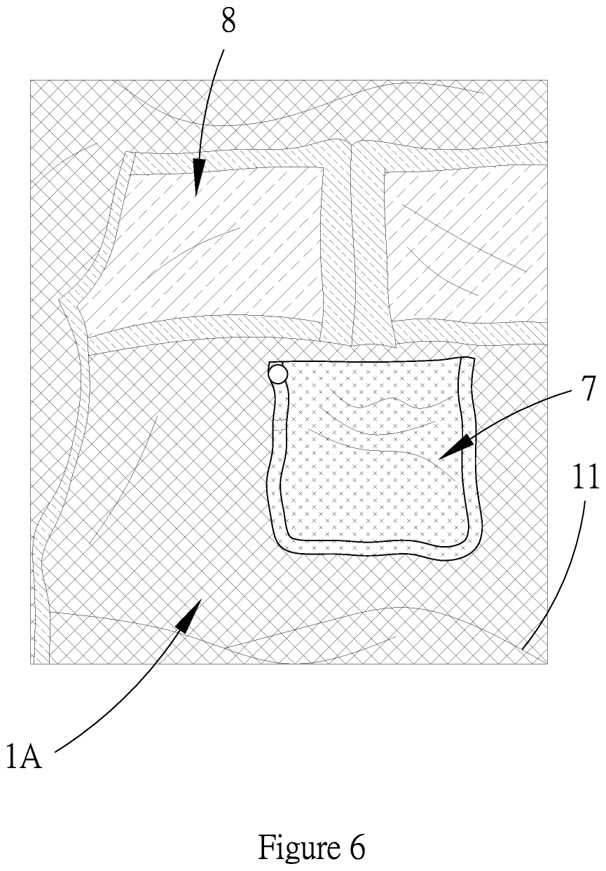

FIG. 6 shows another embodiment of the smart clothing for sensing heart physiological activities. In this embodiment, the clothing body 1A shown in FIG. 6 is different from that in FIG. 1, that is, the clothing body 1A is not inside out in FIG. 6 while the outer surface 11 is shown outside. The clothing body 1A here further includes a restraint 8 mounted on its outer surface 11 at an area opposite to an area where the electrodes 20 of the ECG lead collector 2 are located. The restraint 8 provides a tightening effect at that area to make the electrodes 20 better adhered to human skin, such that even if a user is in a large motion, it would not easily displace the ECG lead collector 2 and affect its sensing function.

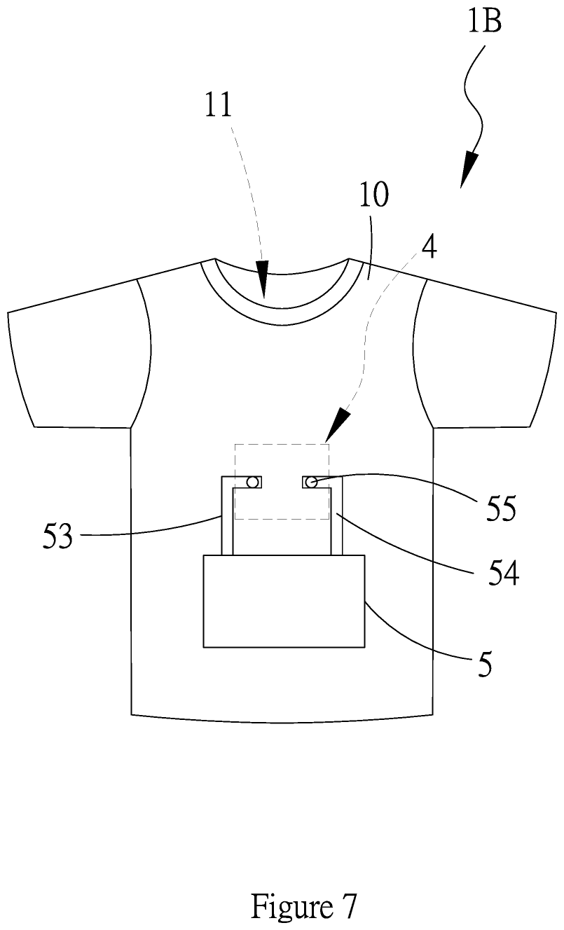

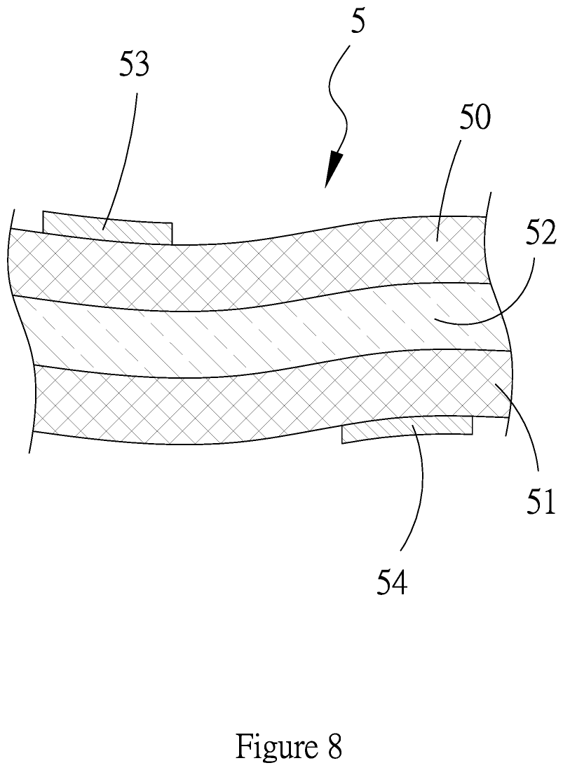

FIG. 7 is a schematic diagram showing appearance of smart clothing applied to sensing lung respiratory conditions in the present invention. The smart clothing in this embodiment is different from the above embodiment shown in FIG. 1 in that, a clothing body 1B in this embodiment has a respiration converter 5 mounted on its contact surface 10. The respiration converter 5 is composed of a multi-layer structure. As shown in FIG. 8, the multi-layer structure includes an upper conductive fabric layer 50, a lower conductive fabric layer 51, an insulation film 52 provided between the upper conductive fabric layer 50 and the lower conductive fabric layer 51, at least one upper wire 53 connected to the upper conductive fabric layer 50, and at least one lower wire 54 connected to the lower conductive fabric layer 51. The respiration converter 5 has its electrical structure as a parallel plate capacitor, which allows a distance between the upper and lower conductive fabric layers 50, 51 to be changed by a user's body cavity undulation when the user breathes, thereby making its capacitance changed, and such change allows the control module 4 to sense the respiratory conditions.

Similar to the above embodiment shown in FIG. 1, there is a conductive connecting element 55 formed at an end of each of the upper wire 53 and the lower wire 54 extended toward the control module 4. The conductive connecting element 55 has a connecting end (not shown here) penetrating the end of the upper wire 53 (or lower wire 54) and the clothing body 1B to be placed on the outer surface 11 of the clothing body 1B. The connecting end of the conductive connecting element 55 is connected to the conductive connecting element of the control module 4. The upper wire 53 and the lower wire 54 are made of conductive fabric, and are used to send capacitance sensed by the upper and lower conductive fabric layers 50, 51 to the control module 4. It should be noted that, the location of the respiration converter 5 is not limited to on the contact surface 10 of the clothing body 1B shown in FIG. 7, but can be on the outer surface 11 of the clothing body 1B. Particularly, the respiration converter 5 is not necessarily in contact with human skin, while it can sense the respiratory conditions by utilizing change in a capacitor structure formed by the upper conductive fabric layer 50 and lower conductive fabric layer 51 according to user's body cavity undulation, such that the respiration converter 5 can alternatively be placed on the outer surface 11 of the clothing body 1B. Further, FIG. 7 shows single multi-layer structure in this embodiment, which is merely to simplify drawings and description, and does not limit implementations of the present invention. It is flexible to mount a plurality of multi-layer structures additionally mounted on the contact surface or the outer surface of the clothing body so as to achieve multiple lung respiratory sensing.

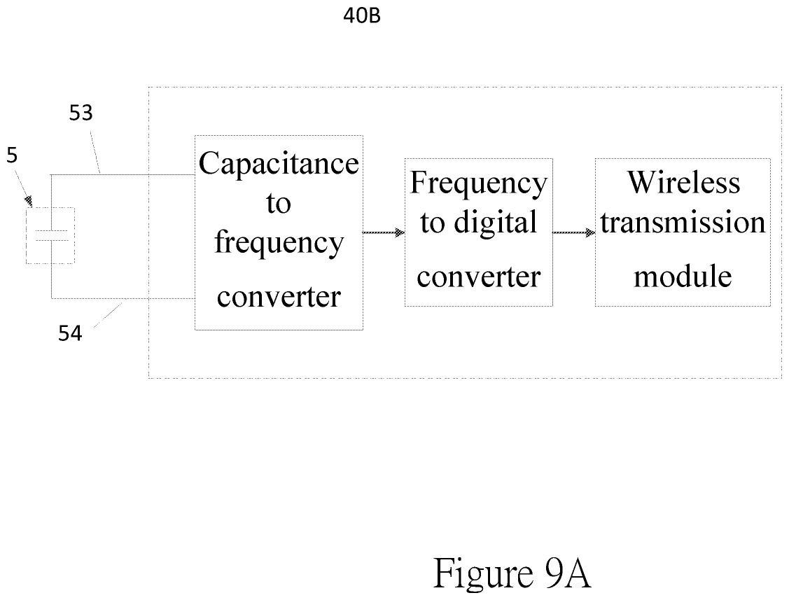

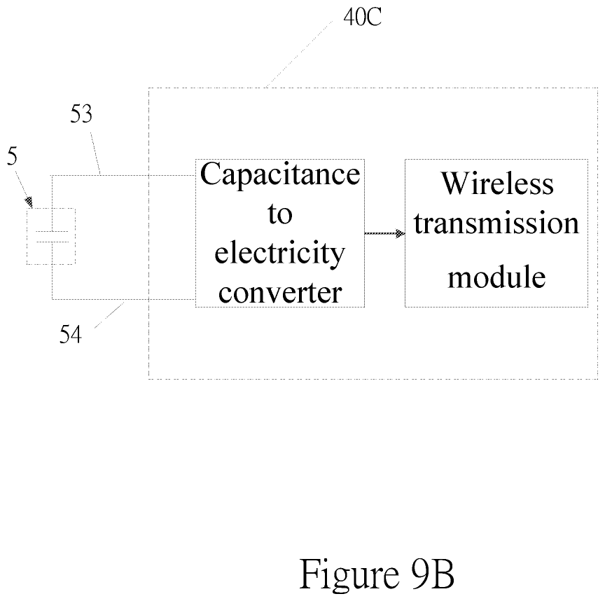

Referring to FIG. 9A and FIG. 9B, which are basic architectural diagrams showing a plurality of functional units of a control module of the smart clothing for sensing lung respiratory conditions according to the present invention, in this embodiment there are two methods for a control module to sense respiratory conditions. One method is shown in FIG. 9A, wherein a control module 40B utilizes a capacitance to frequency converter to convert change of capacitance into frequency change of square wave, then utilizes a frequency to digital converter to digitalize frequency value of the square wave, and lastly transmits the digitalized value to a monitoring device through a wireless transmission module. The other method is shown in FIG. 9B, wherein a control module 40C utilizes a capacitance to electricity converter to convert change of capacitance into voltage, current, electrical quantity or time change (including voltage-sensing, current-sensing, electric charge-sensing and capacitance to time change-sensing method), and allows samples to be taken and transmitted to the monitoring device through a wireless transmission module.

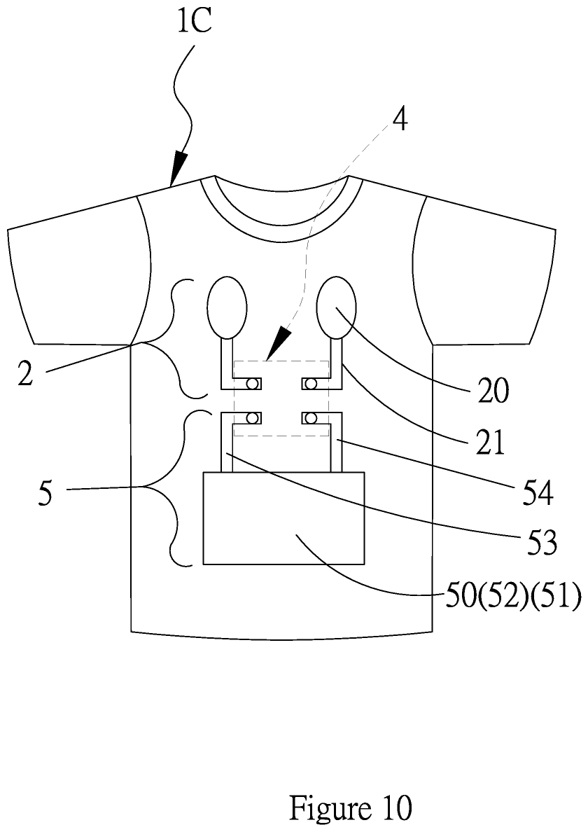

Referring to FIG. 10, which is a schematic diagram showing appearance of smart clothing for sensing heart physiological activities and lung respiratory conditions according to the present invention, this embodiment is different from those in FIG. 1 and FIG. 7 in that, smart clothing 1C according to this embodiment is applicable to both sensing heart physiological activities and sensing lung respiratory conditions. The smart clothing in the present invention is flexibly added with the ECG lead collector 2 and the respiration converter 5 depending on what sensing function a user needs, and can be additionally provided with a plurality of the electrodes 20 made of conductive fabric in response to a required number of ECG leads, as well as conductive fabric wires (includes the lead wires 21, the upper wire 53, the lower wire 54) and conductive connecting elements are mounted on the smart clothing to allow the control module 4 to have a flexible number of transmission interface, such that sensing signals required to be collected are adjustable in quantity, and limitation of designing the conventional smart clothing is thus improved, making it easy to upgrade or alter functions of the smart clothing.

It should be noted that, in order to better adhere the above conductive fabric electrodes or respiration converter to human skin or human body to facilitate sensing, in addition to FIG. 6 with the restraint 8 mounted on the outer surface 11 at a position opposite to the area where the electrodes or respiration converter are located, the clothing body can be made of an elastic material such that when a user wears the clothing body, its elasticity makes the clothing body closely adhered to human body to thereby achieve better sensitivity and give the user more comfort.

It should also be noted that, in another embodiment of smart clothing for sensing heart physiological activities and lung respiratory conditions in the present invention, a zipper (not shown here) can be provided at a collar or waist area of the clothing body at a position opposite to a dressing/undressing direction, to help the user easily put on or take off the clothing body; particularly, the zipper at the collar or waist area is very useful for the user to wear or remove such tight clothing body made of the elastic material.

The examples above are only illustrative to explain principles and effects of the invention, but not to limit the invention. It will be apparent to those skilled in the art that modifications and variations can be made without departing from the scope of the invention. Therefore, the protection range of the rights of the invention should be as defined by the appended claims.

What is claimed is:

1. A Smart clothing for sensing heart physiological activities, including:
a clothing body having a contact surface and an outer surface opposite to the contact surface, wherein the contact surface is for contacting human skin;
an electrocardiogram lead collector including at least one lead collecting structure mounted on the contact surface of the clothing body, wherein the lead collecting structure includes a pair of electrodes, and lead wires respectively connected to the electrodes, with the electrodes and lead wires being made of conductive fabric, wherein each of the lead wires is formed with a first conductive connecting element at an end thereof extended in a predetermined direction, and the first conductive connecting element has a connecting end penetrating the end of each of the lead wires and the clothing body to be placed on the outer surface of the clothing body; and
a control module including a substrate formed with a plurality of functional units and mounted on the outer surface of the clothing body, wherein the substrate is formed with a second conductive connecting element on a side thereof for being connected to the connecting end of the first conductive connecting element, so as to allow the plurality of functional units of the substrate to process heart physiological activity signals sensed by the electrocardiogram lead collector.

2. The smart clothing for sensing heart physiological activities according to claim 1, further including: a spacer provided on part of the contact surface of the clothing body, for covering the lead wires and isolating the lead wires from human skin.

3. The smart clothing for sensing heart physiological activities according to claim 1, wherein the substrate is further formed with a pair of third conductive connecting elements on the side thereof, and the smart clothing further includes:
a respiration converter formed of a multi-layer structure, for outputting capacitance change signals corresponding to human body cavity undulation during breathing, wherein the multi-layer structure includes: an upper conductive fabric layer, a lower conductive fabric layer, an insulation film provided between the upper conductive fabric layer and the lower conductive fabric layer, at least one upper wire connected to the upper conductive fabric layer, and at least one lower wire connected to the lower conductive fabric layer, and the multi-layer structure is mounted on the contact surface or the outer surface of the clothing body, wherein the upper wire and the lower wire are made of conductive fabric, and each of the upper and lower wires is formed with a fourth conductive connecting element at an end thereof extended in the predetermined direction, and the fourth conductive connecting element is connected to the pair of third conductive connecting elements, so as to allow the control module to process the capacitance change signals outputted by the respiration converter and sense human body respiratory conditions.

4. The smart clothing for sensing heart physiological activities according to claim 3, wherein the multi-layer structure is mounted on the contact surface of the clothing body, and the fourth conductive connecting element has an end penetrating the clothing body to be placed on the outer surface of the clothing body.

5. The smart clothing for sensing heart physiological activities according to claim 1, further including: a plurality of electrodes additionally mounted on the contact surface of the clothing body so as to achieve multiple leads electrocardiogram sensing.

6. The smart clothing for sensing heart physiological activities according to claim 3, further including: a spacer provided on the contact surface of the clothing body, for covering the lead wires and the respiration converter, and for isolating the lead wires and the respiration converter from human skin.

7. The smart clothing for sensing heart physiological activities according to claim 1, further including: a container bag attached to the outer surface of the clothing body, for accommodating the control module.

8. The smart clothing for sensing heart physiological activities according to claim 7, further including: a first coupling element formed on the outer surface of the clothing body, wherein the container bag has a movable side formed with a second coupling element for being coupled to the first coupling element, and coupling between the first coupling element and the second coupling element allows the movable side to be lifted or closed in order to detach or mount the control module.

9. The smart clothing for sensing heart physiological activities according to claim 1, further including: a zipper provided at a collar or waist area of the clothing body at a position opposite to a dressing/undressing direction, so as to allow the clothing body to be easily put on or taken off.

10. The smart clothing for sensing heart physiological activities according to claim 1, further including: a restraint mounted on the outer surface of the clothing body at an area opposite to an area where the electrodes of the electrocardiogram lead collector are located, wherein the restraint is for providing a tightening effect at that area to make the electrodes better adhered to human skin.

11. A smart clothing for sensing lung respiratory conditions, including:
a clothing body having a contact surface and an outer surface opposite to the contact surface, wherein the contact surface is for contacting human skin;
a respiration converter formed of a multi-layer structure, for outputting capacitance change signals corresponding to human body cavity undulation during breathing, wherein the multi-layer structure includes: an upper conductive fabric layer, a lower conductive fabric layer, an insulation film provided between the upper conductive fabric layer and the lower conductive fabric layer, at least one upper wire connected to the upper conductive fabric layer, and at least one lower wire connected to the lower conductive fabric layer, and the multi-layer structure is mounted on the contact surface or the outer surface of the clothing body, wherein the upper wire and the lower wire are made of conductive fabric, and each of the upper and lower wires is formed with a first conductive connecting element at an end thereof extended in the predetermined direction; and
a control module including a substrate formed with a plurality of functional units, wherein the substrate is formed with a second conductive connecting element on a side thereof for being connected to the connecting end of the first conductive connecting element, and the substrate is mounted on the contact surface or the outer surface of the clothing body, so as to allow the plurality of functional units of the substrate to process the capacitance change signals outputted by the respiration converter and sense human body respiratory conditions.

12. The smart clothing for sensing lung respiratory conditions according to claim 11, further including: a container bag attached to the outer surface of the clothing body, for accommodating the control module.

13. The smart clothing for sensing lung respiratory conditions according to claim 12, further including: a first coupling element formed on the outer surface of the clothing body, wherein the container bag has a movable side formed with a second coupling element for being coupled to the first coupling element, and coupling between the first coupling element and the second coupling element allows the movable side to be lifted or closed in order to detach or mount the control module.

14. The smart clothing for sensing lung respiratory conditions according to claim 11, further including: a plurality of multi-layer structures additionally mounted on the contact surface or the outer surface of the clothing body so as to achieve multiple lung respiratory sensing.

15. The smart clothing for sensing lung respiratory conditions according to claim 11, further including: a spacer provided on the contact surface of the clothing body, for covering the lead wires and the respiration converter, and for isolating the lead wires and the respiration converter from human skin.

16. The smart clothing for sensing lung respiratory conditions according to claim 11, wherein the multi-layer structure is mounted on the contact surface of the clothing body, and the first conductive connecting element has an end penetrating the clothing body to be placed on the outer surface of the clothing body.

17. The smart clothing for sensing lung respiratory conditions according to claim 11, further including: a zipper provided at a collar or waist area of the clothing body at a position opposite to a dressing/undressing direction, so as to allow the clothing body to be easily put on or taken off.

18. The smart clothing for sensing lung respiratory conditions according to claim 11, wherein the substrate is further formed with a pair of third conductive connecting elements on the side thereof, and the smart clothing further includes an electrocardiogram lead collector, the electrocardiogram lead collector including:
at least one lead collecting structure mounted on the contact surface of the clothing body, wherein the lead collecting structure includes a pair of electrodes, and lead wires respectively connected to the electrodes, with the electrodes and lead wires being made of conductive fabric, wherein each of the lead wires is formed with a fourth conductive connecting element at an end thereof extended in a predetermined direction, and the fourth conductive connecting element has a connecting end penetrating the end of each of the lead wires and the clothing body to be placed on the outer surface of the clothing body, and the fourth conductive connecting element is connected to the pair of third conductive connecting elements, so as to allow the plurality of functional units of the substrate to process heart physiological activity signals sensed by the electrocardiogram lead collector.

19. The smart clothing for sensing lung respiratory conditions according to claim 18, further including: a spacer provided on part of the contact surface of the clothing body, for covering the lead wires and isolating the lead wires from human skin.

20. The smart clothing for sensing lung respiratory conditions according to claim 18, further including: a restraint mounted on the outer surface of the clothing body at an area opposite to an area where the electrodes of the electrocardiogram lead collector are located, wherein the restraint is for providing a tightening effect at that area to make the electrodes better adhered to human skin.

21. The smart clothing for sensing lung respiratory conditions according to claim 18, further including: a plurality of electrodes additionally mounted on the contact surface of the clothing body so as to achieve multiple leads electrocardiogram sensing.
